(19) United States
(12) Patent Application Publication
(10) Pub. No.: US 2025/0082369 A1
(43) Pub. Date: Mar. 13, 2025

(54) SYSTEM FOR AFFIXING AN ORTHOPEDIC COMPONENT TO A BONE USING A POLYAXIAL SCREW

(71) Applicant: Skeletal Dynamics, Inc., Miami, FL (US)

(72) Inventors: Jorge L. Orbay, Miami, FL (US); Robert Sixto, Miami, FL (US); Juergen Kortenbach, Miami, FL (US); Alexander Gil, Miami, FL (US)

(21) Appl. No.: 18/830,270

(22) Filed: Sep. 10, 2024

Related U.S. Application Data
(60) Provisional application No. 63/581,796, filed on Sep. 11, 2023.

(51) Int. Cl. A61B 17/70; A61B 17/86

(52) U.S. Cl. CPC A61B 17/7038; A61B 2017/8655

(57) ABSTRACT

Disclosed is a system suitable for affixing an orthopedic component to a bone using a polyaxial screw comprising an orthopedic component, a screw, and a locking cap; the orthopedic component having a hole with an entry side comprising an internal helical thread and an exit side comprising an internal concave base; the screw having a head comprising a convex base adapted to engage the concave base of the hole, and a convex crown having a plurality of splines; the locking cap comprising a proximal end having a cap socket, a distal end defining a substantially circular contact edge adapted to impinge on the plurality of splines on the crown of the screw when the locking cap is inserted in the hole, and a substantially cylindrical body therebetween having an external helical thread adapted to engage the internal helical thread of the hole.

### CLAIM OF PRIORITY

This application is being filed as a non-provisional patent application under 35 U.S.C. § 111 (a) and 37 CFR § 1.53 (b). This application claims priority under 35 U.S.C. § 119 (e) to U.S. provisional patent application Ser. No. 63/581,796 filed on Sep. 11, 2023, the contents of which are incorporated herein by reference.

### TECHNICAL FIELD

The technical field relates generally to bone fusion devices and systems, and, in particular, to a polyaxial screw and locking cap system for use in orthopedic surgery and similar procedures adapted to prevent rotation or pivoting of the polyaxial screw with respect to an orthopedic plate.

### BACKGROUND

In orthopedic surgery, orthopedic implants are often used when repairing bone fractures. These orthopedic implants include bone plates and screws. To affix an orthopedic bone plate to a bone, screws are often used. These screws can be monoaxial or polyaxial. A monoaxial screw can only be installed along a single predetermined axis, whereas a polyaxial screw allows the screw to be inserted along variable axes.

In some orthopedic surgeries, the insertion of the screw along a single axis is sub-optimal for fixation of the orthopedic bone plate to the bone due to anatomical variations, and screw misplacement can result in complications to the patient. A polyaxial screw allows the screw to be inserted into the bone along an axis selected by the surgeon during a procedure and consequently provides the surgeon with greater flexibility and precision in the placement of the screw.

While polyaxial screws allow for increased flexibility and adjustability during surgery, monoaxial screws often provide greater stability and locking strength. Additionally, polyaxial screw systems are often comprised of several interconnecting parts which can increase the time required by the surgeon to insert and secure the screw during surgery.

Further, compression of the orthopedic implant and fixed angle locking of the screw are important to improve fixation of the orthopedic implant to the bone as well as promote osteointegration. Improved fixation and osteointegration can help support the orthopedic implant and prevent it from backing out. However, current systems do not provide sufficient compression and fixed angle locking while allowing for flexibility and adjustability during surgery.

Therefore, there exists a need for a polyaxial screw system that provides increased locking strength, compression of an orthopedic component, and improved fixation of the orthopedic component while using fewer parts that allow for straightforward and expeditious fixation during surgery.

### SUMMARY

It is among the objects of the present invention to overcome the limitations of the heretofore-known systems by providing inventive features to achieve: a.) superior locking strength; b.) improved fixation of an orthopedic component to a bone; c.) a reduced number of components; d.) improved simplicity and cost-effectiveness in the components utilized; e.) a reduction in the time required to secure a polyaxial screw during a surgical procedure; and f) the ability to compress an implant onto the bone and then lock the position of the screw improving construct strength.

It is an object of the present invention to provide a system suitable for affixing an orthopedic component to a bone using a screw. In one embodiment, the system comprises a substantially rigid orthopedic component having a hole with a diameter; a screw having a longitudinal axis, a leading end, a trailing end, and a cylindrical shaft extending therebetween; and a locking cap comprising a proximal end, a distal end, and a substantially cylindrical body extending therebetween.

The hole of the orthopedic component comprises an entry side and an exit side. The entry side of the hole optionally further comprises an internal helical thread, and the exit side of the hole optionally further comprises an internal concave base. In at least one embodiment, the internal helical thread of the orthopedic component may have an axis that is optionally in a direction normal to an upper surface of the orthopedic component. In other embodiments, the internal helical thread of the orthopedic component may have an axis that is optionally at an angle non-perpendicular to the upper surface of the orthopedic component.

The cylindrical shaft of the screw optionally comprises an external helical thread adapted to engage the bone and a shaft diameter smaller than the hole diameter. The trailing end of the screw comprises a head optionally having a convex base adapted to engage the concave base of the hole, and an optional convex crown. The crown optionally further comprises a screw socket adapted to accept torque from a driver tool, and a plurality of longitudinal splines generally extending in the direction of the longitudinal axis. The plurality of splines form a plurality of longitudinal crests and a plurality of longitudinal valleys on the crown of the screw. In at least some embodiments, the plurality of longitudinal splines may optionally be formed by recesses in the crown. In other embodiments, the plurality of longitudinal splines may optionally be formed by protuberances in the crown.

The body of the locking cap optionally comprises an external helical thread adapted to engage the internal helical thread of the hole of the orthopedic component. The proximal end of the locking cap optionally further comprises a cap socket adapted to accept torque from a driver tool. The distal end of the locking cap may optionally define a substantially circular contact edge. The contact edge is adapted to impinge on the plurality of longitudinal crests on the head of the screw when the locking cap is inserted in the hole.

In at least some embodiments, the contact edge may be made of a material that is harder than the orthopedic component in order to facilitate impinging capability. In further embodiments, the locking cap may optionally have a hardness between 35 and 42 HRC, and the orthopedic component may optionally have a hardness between 20 and 25 HRC.

The optional contact edge of the locking cap may define a substantially circular contact path on the plurality of longitudinal crests when inserted and tightened into the hole after the screw is fully inserted into the hole. In another embodiment in accordance with present invention, the contact edge is optionally castellated to form a plurality of indentations on the contact edge. In at least some embodiments, the contact path may have a length between 20% and 70% of a circumference of the contact edge. In further embodiments, the contact path may have a length between 40% and 60% of the circumference of the contact edge.

In a further embodiment in accordance with the present invention, the system may optionally further comprise a ring. In such embodiments, the system comprises an orthopedic component, a screw, a ring, and a locking cap, and the distal end of the locking cap may further comprise a gland adapted to receive the ring. The ring is adapted to be received by the gland. The ring may comprise a substantially circular contact edge that is adapted to impinge on the plurality of longitudinal crests on the crown of the screw when the locking cap is inserted in the hole. In such embodiments, the contact edge of the ring defines a substantially circular contact path on the plurality of longitudinal crests when inserted into the locking cap and the locking cap is tightened into the hole after the screw is fully inserted into the hole.

In at least some embodiments, the screw may be further adapted to be inserted in a plurality of angles with respect to the orthopedic component. The screw is substantially impeded from changing angles with respect to the orthopedic component when fully inserted into the hole and after the locking cap is inserted into the hole and tightened against the head of the screw.

Although the invention is illustrated and described herein as embodied in connection with a specific orthopedic component, it is nevertheless not intended to be limited to only the details shown, since various modifications and structural changes may be made therein without departing from the spirit of the invention and within the scope and range of equivalents of the claims.

### BRIEF DESCRIPTION OF THE DRAWINGS

FIG. 1 illustrates an isometric view of an assembled system suitable for affixing an orthopedic component to a bone using a screw in accordance with one embodiment.

FIG. 2 illustrates an exploded view of a system suitable for affixing an orthopedic component to a bone using a screw, showing an orthopedic component, a screw, an optional ring, and a locking cap in accordance with one embodiment.

FIGS. 3 and 4 illustrate, respectively, a partial cross-sectional view and a cross-sectional view of an assembled system suitable for affixing an orthopedic component to a bone using a screw, showing additional features in accordance with one embodiment.

FIG. 5 illustrates a cross-sectional view of an orthopedic component in accordance with one embodiment.

FIG. 6 illustrates a side view of a screw in accordance with one embodiment.

FIGS. 7 and 8 illustrate enlarged views of a head of the screw of FIG. 6 showing a plurality of longitudinal splines in accordance with one embodiment.

FIG. 9 illustrates a top view of the head of the screw of FIG. 6 showing additional features in accordance with one embodiment.

FIG. 10 illustrates a cross-sectional view of the relative positioning of a screw and an orthopedic component in accordance with one embodiment.

FIGS. 11 and 12 illustrate, respectively, an isometric view and a side view of a locking cap in accordance with one embodiment.

FIG. 13 illustrates a cross-sectional view of the locking cap of FIGS. 11 and 12 showing additional features in accordance with one embodiment.

FIGS. 14 and 15 illustrate, respectively, a top view and a bottom view of the locking cap of FIGS. 11 and 12 showing additional features in accordance with one embodiment.

FIG. 16 illustrates an isometric view of a ring in accordance with one embodiment.

FIGS. 17 and 18 illustrate, respectively, an exploded view and an exploded cross-sectional view of a locking cap and a ring in accordance with one embodiment.

FIG. 19 illustrates a cross-sectional view of a gland of the locking cap receiving the ring in accordance with one embodiment.

FIGS. 20 and 21 illustrate, respectively, an isometric view and a top view of a substantially circular contact path of a locking cap that contacts a plurality of longitudinal crests on a crown of the head of the screw in accordance with one embodiment, the locking cap shown as transparent for illustrative purposes only.

FIGS. 22 and 23 illustrate, respectively, an isometric view and a side view of the relative positioning of a screw and a locking cap in accordance with one embodiment.

FIG. 24 illustrates a cross-sectional view of the relative positioning of the screw and the locking cap of FIGS. 22 and 23 showing additional features in accordance with one embodiment.

FIG. 25 illustrates an enlarged cross-sectional view of a screw positioned at an angle relative to a locking cap in accordance with another embodiment.

FIG. 26 illustrates an isometric view of a locking cap and a contact edge that is castellated in accordance with another embodiment.

FIGS. 27 and 28 illustrate, respectively, a bottom view and a cross-sectional view of the locking cap and the castellated contact edge of FIG. 26 in accordance with one embodiment.

FIG. 29 illustrates an exploded view of a system suitable for affixing an orthopedic component to a bone using a screw, showing an orthopedic component, a screw, an optional ring, and a locking cap in accordance with one other embodiment.

FIG. 30 illustrates an enlarged cross-sectional view of the relative positioning of a screw received by a hole of an orthopedic component, wherein the hole has an axis different than an axis of an internal helical thread of the hole, and a locking cap not yet received by the hole of the orthopedic component, in accordance with another embodiment.

### DETAILED DESCRIPTION

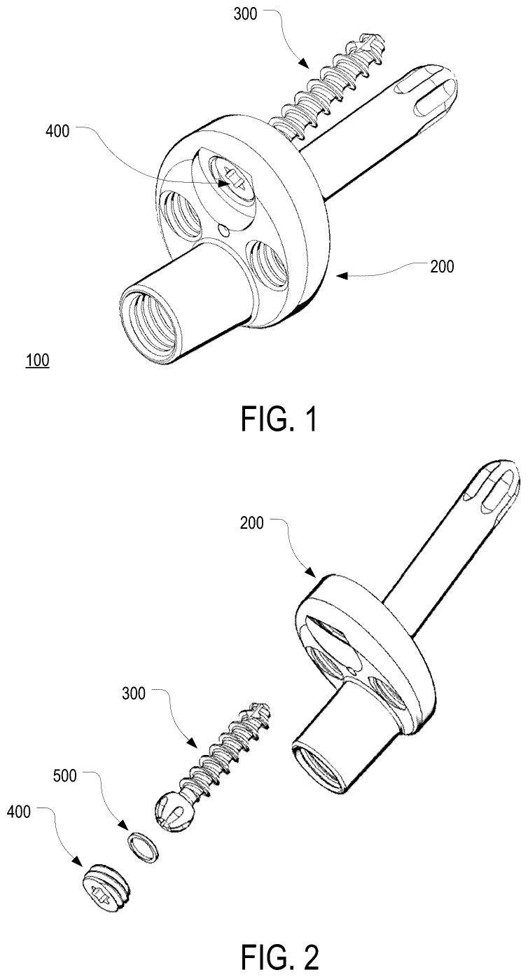

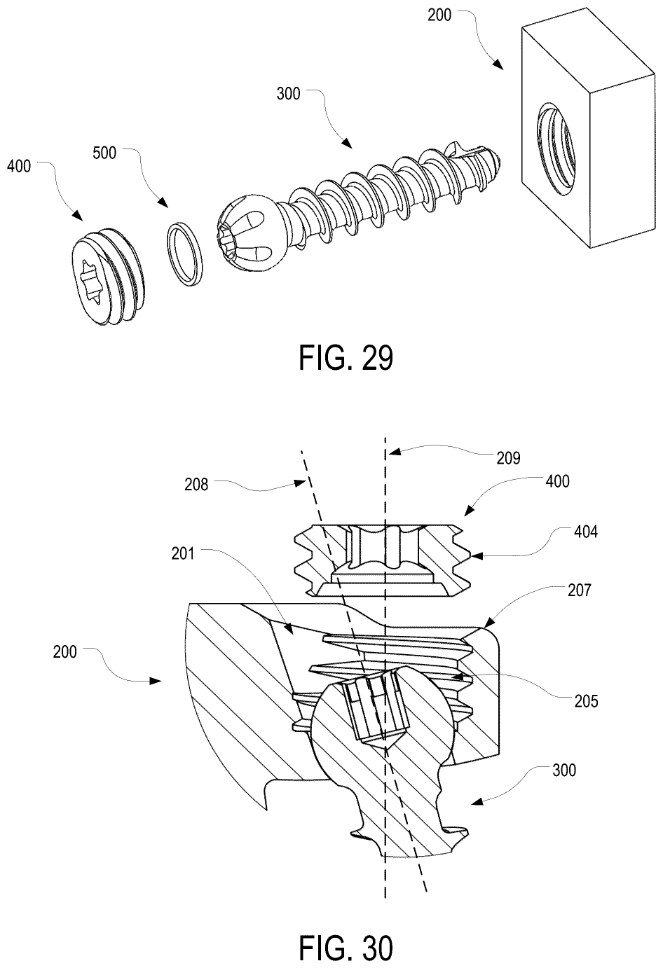

Referring to FIGS. 1, 3, and 4, shown are various views of a system (100) suitable for affixing a substantially rigid orthopedic component (200) to a bone using a screw (300) and a locking cap (400) in accordance with one or more embodiments. The system (100) comprises a substantially rigid orthopedic component (200), a screw (300), and a locking cap (400) in accordance with at least one embodiment. In at least one other embodiment, the system (100) comprises an orthopedic component (200), a screw (300), a locking cap (400), and an optional ring (500). For illustrative purposes only, the system (100) is adapted to an orthopedic component (200) comprising a baseplate for use in shoulder surgery. However, the orthopedic component (200) is not limited to this use or depiction. For example, FIG. 29 depicts another embodiment of an orthopedic component (200). Referring to FIG. 2, an exploded view of the system (100) of FIG. 1, including the optional ring (500), is shown.

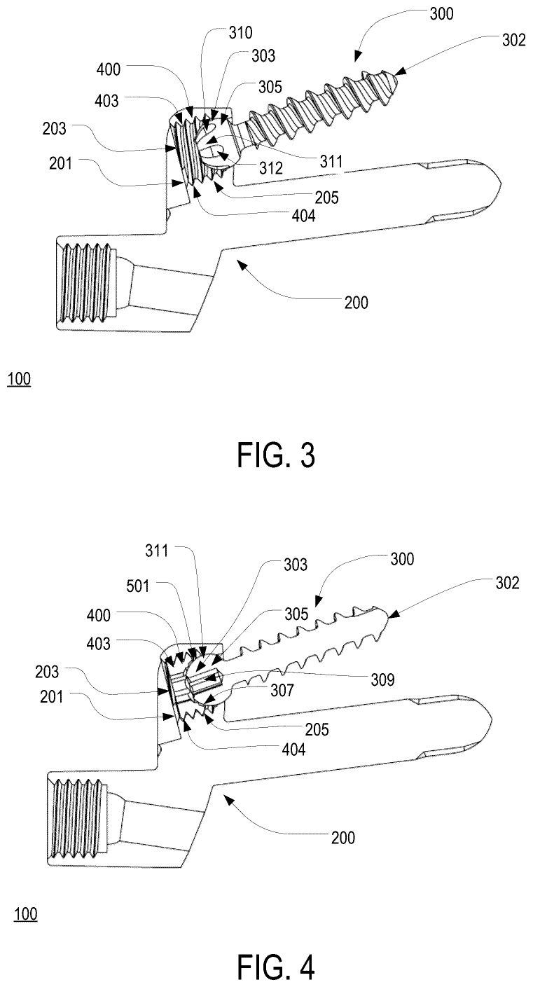

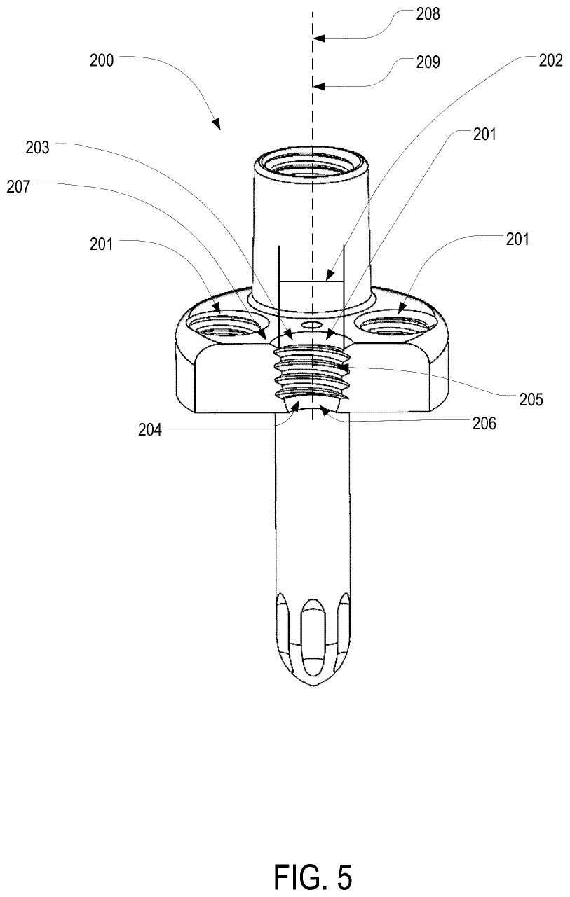

Referring now to FIGS. 4 and 5, a substantially rigid orthopedic component (200) in accordance with at least one embodiment is shown. The orthopedic component (200) comprises at least one hole (201) with a diameter (202). Although the embodiment depicted shows three holes (201), the orthopedic component (200) may comprise one or more holes (201). The hole (201) further comprises an entry side (203) and an exit side (204) through which the screw (300) may travel to reach said bone, as shown assembled in FIG. 3. Referring again to FIG. 5, the entry side (203) of the hole (201) further comprises an internal helical thread (205) adapted to engage the locking cap (400), as shown in FIG. 3. The exit side (204) further comprises an optional internal concave base (206). The internal concave base (206) may optionally be a spherical, parabolic, hyperbolic, or any other irregular curved shape. It is to be understood that various modifications and structural changes may be made to the orthopedic component (200) within the scope and range of equivalents of the orthopedic component (200). For example, FIG. 29 depicts another embodiment of an orthopedic component (200).

Referring to FIGS. 5 and 30, in at least some embodiments, the hole (201) of the orthopedic component may optionally comprise an axis (208) and the internal helical thread (205) may optionally comprise another axis (209). In some embodiments, such as the one depicted in FIG. 5, the axis (208) of the hole (201) and the axis (209) of the internal helical thread (205) may optionally align and be the same. In other embodiments, such as the one depicted in FIG. 30, the axis (208) of the hole (201) and the axis (209) of the internal helical thread (205) may optionally not be the same and may not align. Referring further to FIG. 30, in some embodiments, the axis (209) of the internal helical thread (205) may optionally be in a direction normal to an upper surface (207) of the orthopedic component (200). Embodiments having a hole (201) axis (208) and internal helical thread (205) axis (209) that are not the same help facilitate installation of the locking cap (400). These optional differing axes (208, 209) may allow the hole (201) axis (208) to be in the direction of an anatomical feature, while the internal helical thread (205) axis (209) may optionally be in a direction normal to the upper surface (207) such that the locking component (200) can be aligned with the upper surface (207) of the orthopedic component (200) upon insertion into the hole (201) of the orthopedic component (200). In other embodiments, the axis (209) of the internal helical thread (205) may be angled, or in a direction non-perpendicular, with respect to the upper surface (207) of the orthopedic component (200). In some embodiments, such as the one depicted in FIG. 30, the axis (208) of the hole (201) may optionally be angled, or in direction non-perpendicular, with respect to the upper surface (207) of the orthopedic component. The non-perpendicular angle of the hole (201) axis (208) with respect to the upper surface (207) facilitates insertion of the screw (300) at an angle non-perpendicular to the upper surface (207). In other embodiments, such as the one depicted in FIG. 5, the axis (208) of the hole (201) may be in direction normal to the upper surface (207) of the orthopedic component (200).

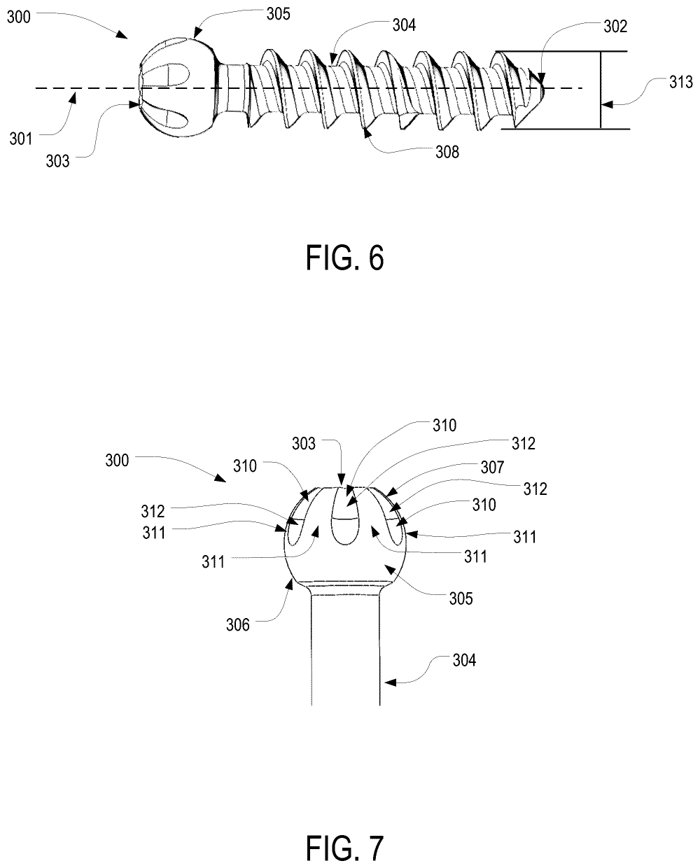

Now referring to FIGS. 6 through 9, various views of a screw (300) in accordance with at least one embodiment are shown. Referring to FIG. 6, the screw (300) comprises a longitudinal axis (301) extending from a trailing end (303) to a leading end (302) of the screw. A cylindrical shaft (304) extends between the leading end (302) and the trailing end (303) of the screw (300). The shaft (304) further comprises a shaft diameter (313) that is smaller than the hole diameter (202) of the orthopedic component (200) shown in FIG. 5, and may optionally comprise an external helical thread (308) adapted to engage a bone.

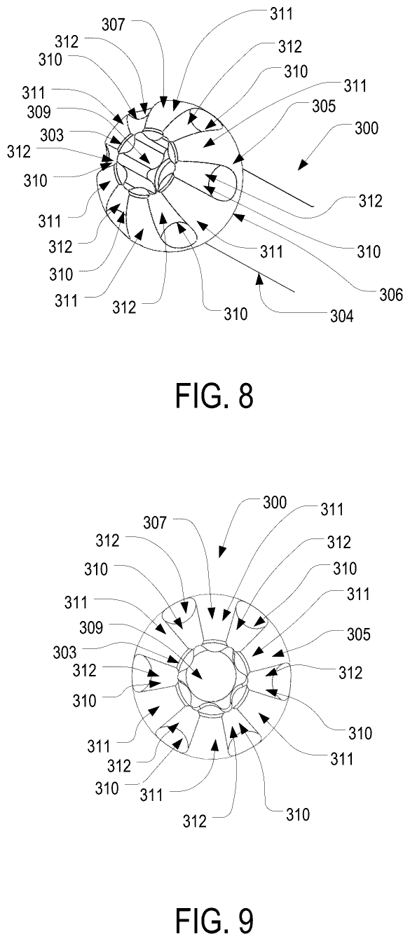

Referring to FIG. 7, the trailing end (303) of the screw (300) comprises a head (305). The head (305) may optionally further comprise a convex base (306) and a convex crown (307). The convex base (306) may optionally be a spherical, parabolic, hyperbolic, or any other irregular curved shape. Similarly, the convex crown (307) may optionally be a spherical, parabolic, hyperbolic, or any other irregular curved shape. As further shown in FIGS. 6 through 9, the crown (307) may optionally comprise a screw socket (309) adapted to accept torque from a driver tool (not shown), and a plurality of longitudinal splines (310) generally extending in the direction of the longitudinal axis (301) shown in FIG. 6. In one embodiment, the plurality of longitudinal splines (310) are formed by recesses in the crown (307). In at least one other embodiment, the plurality of longitudinal splines (310) are formed by protuberances in the crown (307). In some embodiments, the crown (307) may comprise between 4 and 10 splines (310). In at least one embodiment, such as the embodiment depicted in FIGS. 8 and 9, the crown (307) comprises 6 splines (310).

Referring again to FIGS. 6 through 9, the plurality of longitudinal splines (310) results in a plurality of longitudinal crests (311) in the relatively smooth space between splines, and a plurality of longitudinal valleys (312) at the lowest points of each spline (310), generally extending in the direction of the longitudinal axis (301). Although in the embodiment shown the plurality of longitudinal splines (310) are all depicted as the same size and positioned at equal distance to each other, the plurality of longitudinal splines (310) may also be of varying sizes and positioned at varying distances with respect to one another. Similarly, the plurality of longitudinal crests (311) and the plurality of longitudinal valleys (312) may also vary in size and spacing from one another. It should also be observed that in some embodiments the splines (310) may comprise protuberances, not recesses, and that the crests (311) may therefore be formed at the highest point of the splines, with the valleys (312) formed in the smooth space between the splines (310).

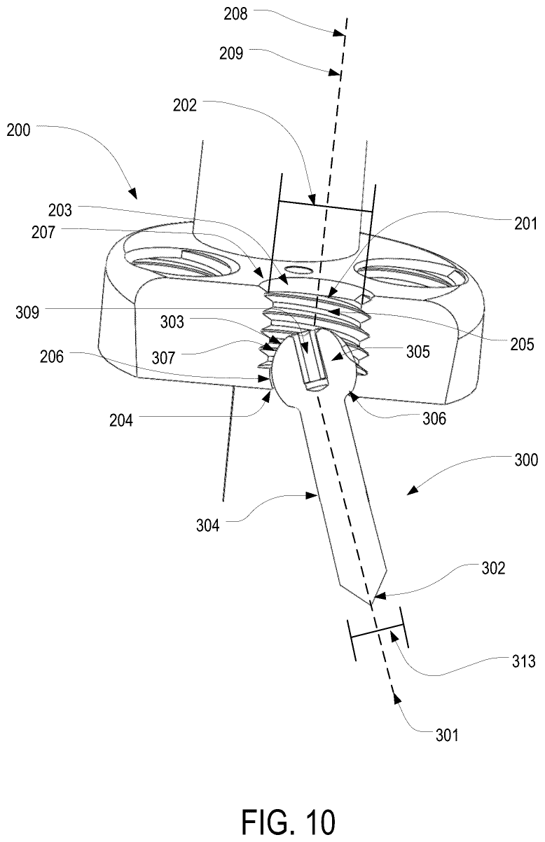

Referring next to FIG. 10, in at least one embodiment, the leading end (302) of the screw (300) enters initially through the entry side (203) of the hole (201) of the orthopedic component (200) and exits through the exit side (204) of the hole (201). The trailing end (303) of the screw (300) follows partially through the hole (201) until the convex base (306) of the head (305) on the screw (300) engages the concave base (206) of the hole (201). Due to the mating spherical interface between the convex base (306) of the screw (300), and the concave base (206) of the hole (201), the screw (300) is adapted to be inserted in a plurality of angles with respect to the orthopedic component (200). The interface between the convex base (306) of the screw (300) and the concave base (206) of the hole (201) may optionally be other complementary concave/convex shapes, including but not limited to spherical, parabolic, hyperbolic, or any other irregular curved shapes.

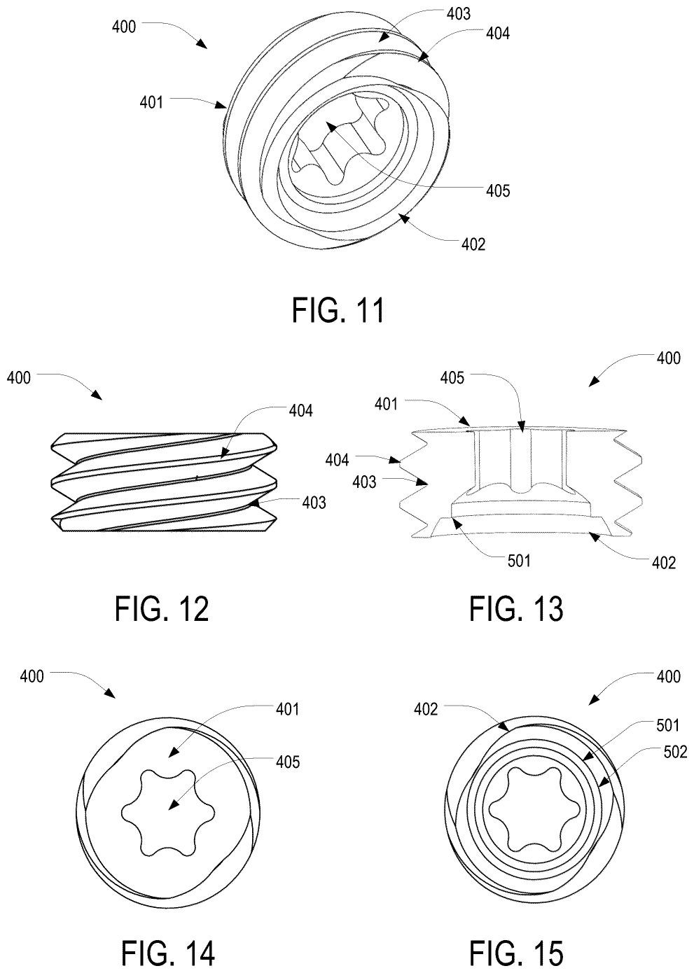

Referring to FIGS. 11 through 15, shown are various views of a locking cap (400) in accordance with at least one embodiment. As shown in FIGS. 11, 12, and 13, the locking cap (400) comprises a proximal end (401), a distal end (402), and a substantially cylindrical body (403) extending therebetween. Referring to FIG. 12, the body (403) may optionally further comprise an external helical thread (404) adapted to engage the internal helical thread (205) of the hole (201) on the orthopedic component (200) (See FIG. 3). Referring back to FIG. 4, the locking cap (400) is shown after having engaged with the orthopedic component (200) and the head (305) of the screw (300). The screw (300) is substantially impeded from changing angles with respect to the orthopedic component (200) when fully inserted into the hole (201) and after the locking cap (400) is inserted into the hole and tightened against the head (305) of the screw (300), as shown in FIG. 4.

Referring to FIGS. 13 and 14, the proximal end (401) of the locking cap (400) may optionally further comprise a cap socket (405) adapted to accept torque from a driver tool (not shown). Referring now to FIGS. 13 and 15, the distal end (402) of the locking cap (400) may optionally comprise a substantially circular contact edge (501).

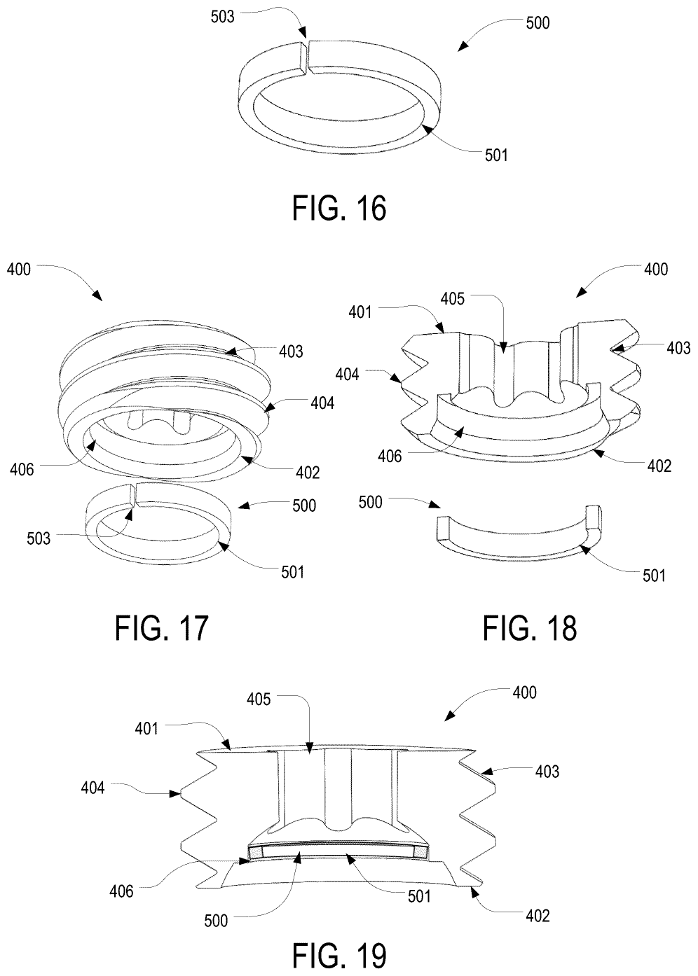

Referring again to FIG. 2, in at least one other embodiment, the system comprises an orthopedic component (200), a screw (300), a locking cap (400), and an optional ring (500), and the distal end (402) of the locking cap (400) comprises a gland (406), instead of the contact edge (501), as shown in FIGS. 17 through 19. Referring to FIG. 16, a ring (500) in accordance with at least one embodiment is shown. Referring again to FIGS. 17 and 18, an exploded view and an exploded cross-sectional view, respectively, are shown of the ring (500) and the locking cap (400) with the gland (406) in accordance with one embodiment. Referring again to FIG. 16, the ring (500), instead of the locking cap (400), comprises the substantially circular contact edge (501). As shown in FIG. 19, the gland (406) of the locking cap (400) is adapted to receive the ring (500).

Referring again to FIGS. 16 and 17, the ring may comprise a slit (503) adapted to permit the ring (500) to compress when inserted into the gland (406) and to be removably secured within the locking cap (400) by a spring action resulting from such compression.

Referring to FIGS. 13 and 16, the contact edge (501) may be relatively sharp and adapted to cut into, or deform, the crown (307) when tightened with sufficient force against the head (305) of the screw (300) (not shown).

In some embodiments, the locking cap (400) and the ring (500) may be made of CoCr. In other embodiments, the locking cap (400) may be made of CoCr and the ring (500) of Ti15Mo. In further embodiments having a ring (500) and a locking cap (400), the locking cap (400) may optionally be made of any Titanium Alloy, and the ring (500) may optionally be made of CoCr, stainless steel, etc. In embodiments without the ring (500), the locking cap (400) may be made of CoCr. However, any other suitable materials may be used. Ideally, the material of the contact edge (501) should be harder than the material of the head (305) and/or crown (307) of screw (300). In at least some embodiments, the screw (300) may be made of a titanium alloy, such as, for example, Ti6Al4V. In other embodiments, the locking cap (400) may be made of Ti15Mo with no ring (500). In such embodiments, the screw may still be made of a titanium alloy, such as, Ti6Al4V. Further, the contact edge (501) may be made of a material that is harder than the orthopedic component (200) in order to facilitate the impinging capability.

In further embodiments, the locking cap (400) has a hardness between 35 and 42 HRC, and the orthopedic component (200) has a hardness between 20 and 25 HRC.

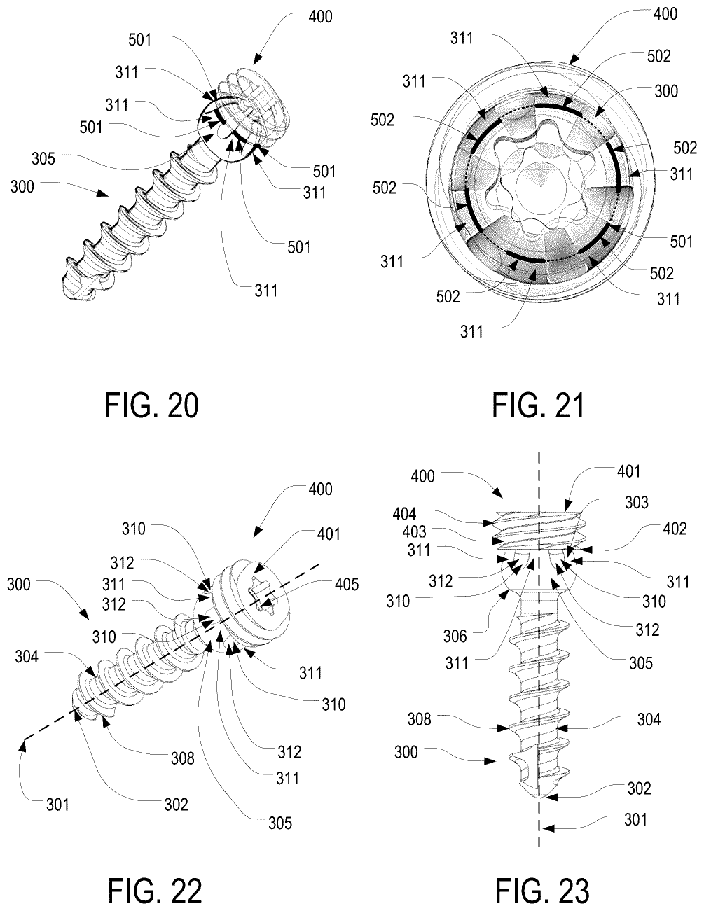

Referring now to FIGS. 20 and 21, when the locking cap (400) is tightened against the head (305) of the screw (300), the contact edge (501) defines a substantially circular contact path (502). In at least some embodiments, the substantially circular contact path (502) may optionally be comprised of multiple segments that contact only the plurality of longitudinal crests (311) on the crown (307), as shown in FIG. 21. In some embodiments the combined length of the multiple segments of contact path (502) is between 20% and 70% of the total circumference of contact edge (501). In at least some embodiments, the combined length of the multiple segments of contact path (502) is a length between 40% and 60% of the circumference of the contact edge (501). In at least one further embodiment, the combined length of the multiple segments of the contact path (502) is approximately equal to 50% of the circumference of the contact edge (501).

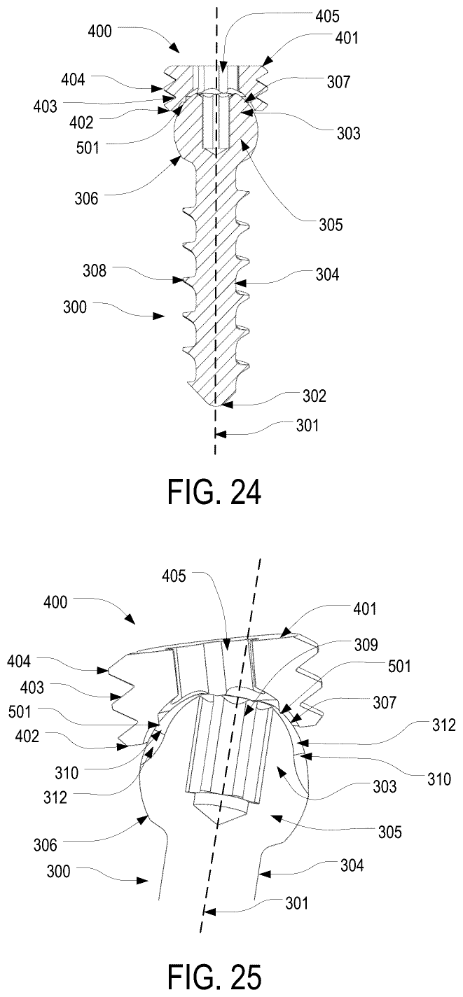

Referring next to FIGS. 22 through 24, shown are various views of the screw (300) engaged with the locking cap (400) in accordance with at least one embodiment. As further shown in FIGS. 3 and 4, the contact edge (501) is adapted to impinge on the plurality of longitudinal crests (311) on the crown (307) of the screw (300) when the locking cap (400) is inserted and tightened into the hole (201) of the orthopedic component (200).

It should be noted that the screw (300) and the locking cap (400) need not be in axial alignment in order to result in a locked arrangement. The angle of alignment between the screw (300) and the locking cap (400) depends on the final angle of the screw (300) with respect to the plane of the hole (201) of the orthopedic component (200). In FIGS. 22, 23, and 24, for example, the screw (300) and the locking cap (400) are shown in axial alignment because the screw (300) has an angle of insertion that is substantially perpendicular to the plane of the hole (201) (not shown). However, in FIG. 25 the angle of insertion of the screw (300) with respect to the plane of the hole (201) is not perpendicular (see, for example, FIG. 30), resulting in no axial alignment between the locking cap (400) and the screw (300).

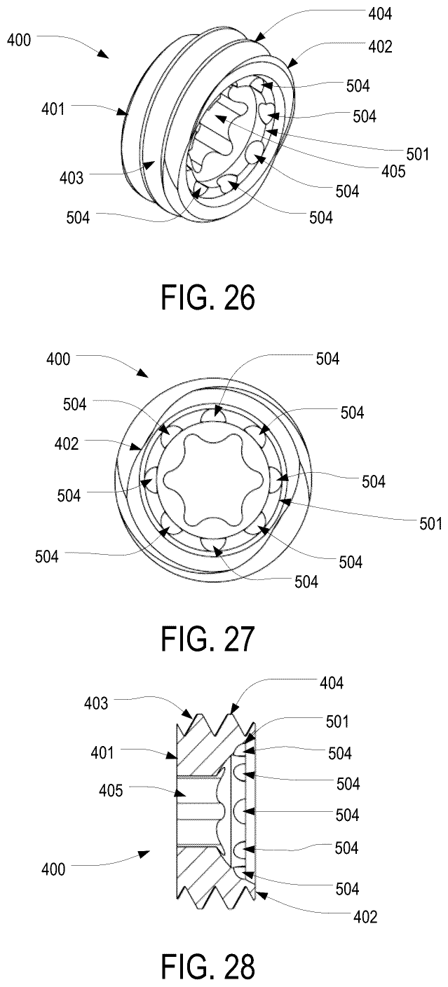

Referring to FIGS. 26, 27, and 28, in another embodiment, the contact edge (501) may optionally be castellated forming a plurality of indentations (504) along the contact edge (501).

Any reference in this specification to “one embodiment,” “an embodiment,” an “example embodiment,” etc., means that a particular feature, structure, or characteristic described in connection with the implementation is included in at least one embodiment of the invention. The appearances of such phrases in various places in the specification are not necessarily referring to the same embodiment. In addition, any elements or limitations of any invention or embodiment thereof disclosed herein can be combined with any and/or all other elements or limitations (individually or in any combination) or any invention or embodiment thereof disclosed herein, and all such combinations are contemplated with the scope of the invention without limitation thereto.

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

Although described above in connection with particular configurations, these descriptions are not intended to be limiting as various modifications may be made therein without departing from the spirit of the invention and within the scope and range of equivalents of the described embodiments. Encompassed embodiments of the present invention can be used in all surgical applications calling for polyaxial screws.

While a number of embodiments of the present invention have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A system suitable for affixing an orthopedic component to a bone using a screw, the system comprising:
a substantially rigid orthopedic component having a hole with a diameter, the hole comprising an entry side and an exit side, the entry side of the hole comprising an internal helical thread, and the exit side of the hole comprising an internal concave base;
a screw having a longitudinal axis, a leading end, a trailing end, and a cylindrical shaft extending therebetween, the trailing end of the screw comprising a head having a convex base adapted to engage the concave base of the hole and a convex crown, the cylindrical shaft comprising an external helical thread adapted to engage said bone and having a shaft diameter smaller than the hole diameter;
the crown comprising a screw socket adapted to accept torque from a driver tool, and a plurality of longitudinal splines generally extending in the direction of the longitudinal axis, the plurality of splines forming a plurality of longitudinal crests and a plurality of longitudinal valleys on the crown of the screw;
a locking cap comprising a proximal end, a distal end, and a substantially cylindrical body extending therebetween, the body comprising an external helical thread adapted to engage the internal helical thread of the hole, the proximal end comprising a cap socket adapted to accept torque from a driver tool, and the distal end defining a substantially circular contact edge, the contact edge adapted to impinge on the plurality of longitudinal crests on the head of the screw when the locking cap is inserted in the hole;
wherein the contact edge of the locking cap defines a substantially circular contact path on the plurality of longitudinal crests when inserted and tightened into the hole after the screw is fully inserted into the hole.

2. The system of claim 1 wherein:
the screw is adapted to be inserted in a plurality of angles with respect to the orthopedic component; and
the screw is substantially impeded from changing angles with respect to the orthopedic component when fully inserted into the hole and after the locking cap is inserted into the hole and tightened against the head of the screw.

3. The system of claim 1 wherein the plurality of longitudinal splines are formed by recesses in the crown.

4. The system of claim 1 wherein the plurality of longitudinal splines are formed by protuberances in the crown.

5. The system of claim 1 wherein the contact path has a length between 20% and 70% of a circumference of the contact edge.

6. The system of claim 1 wherein the contact path has a length between 40% and 60% of a circumference of the contact edge.

7. The system of claim 1 wherein the contact edge is castellated forming a plurality of indentations on the contact edge.

8. The system of claim 1 wherein:
the locking cap has a hardness between 35 and 42 HRC; and
the orthopedic component has a hardness between 20 and 25 HRC.

9. The system of claim 1 wherein the internal helical thread of the orthopedic component has an axis in a direction normal to an upper surface of the orthopedic component.

10. The system of claim 1 wherein the internal helical thread of the orthopedic component has an axis at an angle non-perpendicular to an upper surface of the orthopedic component.

11. A system suitable for affixing an orthopedic component to a bone using a screw, the system comprising:
a substantially rigid orthopedic component having a hole with a diameter, the hole comprising an entry side and an exit side, the entry side of the hole comprising an internal helical thread, and the exit side of the hole comprising an internal concave base;
a screw having a longitudinal axis, a leading end, a trailing end, and a cylindrical shaft extending therebetween, the trailing end of the screw comprising a head having a convex base adapted to engage the concave base of the hole and a convex crown, the cylindrical shaft comprising an external helical thread adapted to engage said bone and having a shaft diameter smaller than the hole diameter;
the crown comprising a screw socket adapted to accept torque from a driver tool, and a plurality of longitudinal splines generally extending in the direction of the longitudinal axis, the plurality of splines forming a plurality of longitudinal crests and a plurality of longitudinal valleys on the crown of the screw;
a locking cap comprising a proximal end, a distal end, and a substantially cylindrical body extending therebetween, the body comprising an external helical thread adapted to engage the internal helical thread of the hole, the proximal end comprising a cap socket adapted to accept torque from a driver tool, the distal end comprising a gland; and
a ring adapted to be received by the gland of the locking cap, the ring defining a substantially circular contact edge, the contact edge adapted to impinge on the plurality of longitudinal crests on the head of the screw when the ring and the locking cap are inserted in the hole;
wherein the contact edge of the ring defines a substantially circular contact path on the plurality of longitudinal crests when the ring and the locking cap are inserted and tightened into the hole after the screw is fully inserted into the hole.

12. The system of claim 11 wherein:
the screw is adapted to be inserted in a plurality of angles with respect to the orthopedic component; and
the screw is substantially impeded from changing angles with respect to the orthopedic component when fully inserted into the hole and after the ring and the locking cap are inserted into the hole and tightened against the head of the screw.

13. The system of claim 11 wherein the plurality of longitudinal splines are formed by recesses in the crown.

14. The system of claim 11 wherein the plurality of longitudinal splines are formed by protuberances in the crown.

15. The system of claim 11 wherein the contact path has a length between 20% and 70% of a circumference of the contact edge.

16. The system of claim 11 wherein the contact path has a length between 40% and 60% of a circumference of the contact edge.

17. The system of claim 11 wherein the contact edge is castellated forming a plurality of indentations on the contact edge.

18. The system of claim 11 wherein:
the locking cap has a hardness between 35 and 42 HRC; and
the orthopedic component has a hardness between 20 and 25 HRC.

19. The system of claim 11 wherein the internal helical thread of the orthopedic component has an axis in a direction normal to an upper surface of the orthopedic component.

20. The system of claim 11 wherein the internal helical thread of the orthopedic component has an axis at an angle non-perpendicular to an upper surface of the orthopedic component.
